(19) United States
(12) Patent Application Publication
(10) Pub. No.: US 2012/0215046 A1
(43) Pub. Date: Aug. 23, 2012

(54) Alkylation Process and Catalysts for Use Therein

(75) Inventor: James R. Butler, Spicewood, TX (US)

(73) Assignee: FINA TECHNOLOGY, INC., Houston, TX (US)

(21) Appl. No.: 13/352,341

(22) Filed: Jan. 18, 2012

Related U.S. Application Data
(60) Provisional application No. 61/444,172, filed on Feb. 18, 2011.

(51) Int. Cl. C07C 2/66; B82Y 30/00

(52) U.S. Cl. 585/467; 977/700; 977/902

(57) ABSTRACT

Disclosed is a method for aromatic conversion that includes contacting an alkene and an aromatic hydrocarbon with a nanocrystalline zeolite catalyst disposed within a reactor under alkylation conditions, wherein the nanocrystalline zeolite catalyst includes at least one zeolitic material and producing a product stream having a monoalkyl aromatic hydrocarbon.

### CROSS-REFERENCE TO RELATED APPLICATIONS

This application is a non-provisional of U.S. Provisional Application Ser. No. 61/444,172 filed Feb. 18, 2011.

### FIELD

Embodiments described herein generally relate to the production of alkyl aromatic hydrocarbons through alkylation reactions. Additionally, the embodiments relate to alkylation catalysts used in such reactions.

### BACKGROUND

Alkylation reactions generally involve contacting a first aromatic compound with an alkylation agent in the presence of a catalyst to form a second aromatic compound. One important alkylation reaction is the reaction of benzene with ethylene in the production of ethylbenzene. The ethylbenzene can then be dehydrogenated to form styrene.

Styrene is an important monomer used in the manufacture of many polymers. Efforts are continually underway to improve catalysts for such process and reduce by-product formation.

### SUMMARY OF THE INVENTION

Disclosed is a method for aromatic conversion that includes contacting an alkene and an aromatic hydrocarbon with a nanocrystalline zeolite catalyst disposed within a reactor under alkylation conditions, wherein the nanocrystalline zeolite catalyst includes at least one zeolitic material and producing a product stream having a monoalkyl aromatic hydrocarbon.

In an embodiment the selectivity for the monoalkyl aromatic hydrocarbon is at least 92 mass percent, optionally at least 95 mass percent, or optionally at least 97 mass percent.

In an embodiment the product stream has less than 5 mass percent of a polyalkyl aromatic hydrocarbon, optionally less than 4 mass percent, or optionally less than 3 mass percent.

### BRIEF DESCRIPTION OF DRAWINGS

FIG. 1 is a schematic block diagram of an embodiment of an alkylation/transalkylation process.

FIG. 2 is a schematic illustration of a parallel reactor system that can be used for an alkylation process.

FIG. 3 illustrates one embodiment of an alkylation reactor with a plurality of catalyst beds.

### DETAILED DESCRIPTION

### Introduction and Definitions

Embodiments described herein generally utilize a nanocrystalline zeolite catalyst for aromatic conversion of an aromatic hydrocarbon to form an alkyl aromatic product having fewer polyalkyl aromatic byproducts than with traditional alkylation catalysts.

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions when the information in this patent is combined with available information and technology.

Various terms as used herein are shown below. To the extent a term used in a claim is not defined below, it should be given the broadest definition skilled persons in the pertinent art have given that term as reflected in printed publications and issued patents at the time of filing. Further, unless otherwise specified, all compounds described herein may be substituted or unsubstituted and the listing of compounds includes derivatives thereof.

Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations.

The term “aromatic” may have a scope recognized by one skilled in the art, which includes alkyl substituted and unsubstituted mono- and polynuclear hydrocarbons.

The term “substituted”, in reference to alkylatable aromatic hydrocarbons, includes aromatic compounds that possess at least one hydrogen atom directly bonded to the aromatic nucleus.

The term “polyalkyl aromatic hydrocarbon” refers to aromatic hydrocarbons having more than one alkyl group, including dialkyl and diarylalkyl aromatic compounds.

The terms “transalkylation”, “transalkylating”, and variants thereof generally refer to the exchange of alkyl substituent groups between aromatic hydrocarbons.

The term “zeolitic material” includes a molecular sieve having a framework.

The term “nanocrystalline zeolite catalyst” refers to a catalyst having at least one zeolitic material with a particle size smaller than 600 nm.

The term “particle size” refers to either the size of each discrete crystal (i.e., crystal) of the zeolitic material or the size of an agglomeration of particles (i.e., crystallite) within the zeolitic material.

The term “activity” refers to the weight of product produced per weight of the catalyst used in a method at a standard set of conditions per unit of time.

The term “selectivity” refers to the percent of monoalkyl aromatic hydrocarbon produced from the reacted alkene. For example:

S_{EB}=selectivity of benzene to EB=EBₒᵤₜ/Bz_{converted}

The term “aromatic conversion” includes alkylation of an aromatic hydrocarbon to form alkyl aromatic hydrocarbon product.

Embodiments described herein utilize a nanocrystalline zeolite catalyst within one or more catalysts beds of an alkylation process. The nanocrystalline zeolite catalyst is made of at least one zeolitic material.

Suitable zeolitic materials may include zeolite Y (including rare earth exchanged zeolite Y), zeolite X (including rare earth exchanged zeolite X), MCM-22, MCM-36, MCM-49, zeolite beta, ZSM-4, ZSM-12, ZSM-20, ZSM-38, MCM-56, faujasite, mordenite, PSH-3, SSZ-25, ERB-1, ITQ-1, ITQ-2, and combinations thereof. The zeolitic materials may comprise large pores, with the zeolitic materials having a Constraint Index less than 2, for example. Suitable large pore zeolitic materials include zeolite beta, zeolite Y, Ultrastable Y (USY), Dealuminized Y (Deal Y), rare earth exchanged Y (REY), mordenite, ZSM-3, ZSM-4, ZSM-18, and ZSM-20. Mordenite is a naturally occurring material but is also available in synthetic forms, such as TEA-mordenite (i.e., synthetic mordenite prepared from a reaction mixture comprising a tetraethylammonium directing agent).

Zeolite X may have a silicon to aluminum molar ratio of from about 1:1 to about 1.7:1, and zeolite Y may have a silicon to aluminum molar ratio of greater than about 1.7:1, for example.

Silicate-based zeolitic materials, such as faujasites and mordenites, may be formed of alternating SiO₂ and MO_{X} tetrahedra, where M is an element selected from the Groups 1 through 16 of the Periodic Table. Such formed zeolitic materials may have 4, 6, 8, 10, or 12-membered oxygen ring channels, for example.

The nanocrystalline zeolite catalyst generally includes from about 1 wt. % to about 99 wt. %, or from 3 wt. % to about 90 wt. % or from about 4 wt. % to about 80 wt. % zeolitic material, for example.

The zeolitic material of the nanocrystalline zeolite catalyst may have a particle size of smaller than about 600 nanometers (nm). For example, the particle size may be less than 500 nm, or less than 300 nm, or less 100 nm, or less than 50 nm or less than 25 nm, for example. In one or more embodiments, the particle size is from 25 nm to 300 nm, or from 50 nm to 100 nm or from 50 nm to 75 nm, for example.

The nanocrystalline zeolite catalyst may further include a support material. Suitable support materials may include silica, alumina, aluminosilica, titania, zirconia, silicon carbide and combinations thereof, for example. In one or more embodiments, the nanocrystalline zeolite catalyst includes from about 5 wt. % to about 97 wt. %, or from about 5 wt. % to about 95 wt. % or from about 7 wt. % to about 90 wt. % support material, for example.

The zeolitic material may be supported by methods known to one skilled in the art. For example, such methods may include impregnating a solid, porous alumino silicate particle or structure with a concentrated aqueous solution of an inorganic micropore-forming directing agent through incipient wetness impregnation.

Alternatively, the zeolitic material may be admixed with a support material, for example. It is further contemplated that the zeolitic material may be supported in-situ with the support material or extruded, for example.

It is further contemplated that such support methods may include layering the zeolitic material onto the support material, for example. It is further contemplated that such support methods may include the utilization of zeolite membranes, for example.

In one embodiment, the zeolitic material is supported by incipient wetness impregnation. Such method generally includes dispersing the zeolitic material in a diluent, such as methanol, to yield individual crystals. A support material may then be added to the solution and mixed until dry.

In yet another embodiment, the zeolitic material may be supported by forming a mini extrusion batch utilizing a support material in combination with the zeolitic material to form extrudates. A multitude of different extrudate shapes are possible, including but not limited to cylinders, cloverleaf, dumbbell and symmetrical and asymmetrical polylobates. Typical diameters of extrudates are 1.6 mm ( 1/16 in.) and 3.2 mm (⅛ in.). The extrudates may be further shaped to any desired form, such as spheres, by any means known to one skilled in the art.

In one or more embodiments, the nanocrystalline zeolite catalyst may include one or more inorganic oxides including but not limited to beryllia, germania, vanadia, tin oxide, zinc oxide, iron oxide and cobalt oxide; non-zeolitic molecular sieves; and spinels such as MgAl₂O₄, FeAl₂O₄, ZnAl₂O₄, CaAl₂O₄, and other like compounds having the formula MO—Al₂O₃ where M is a metal having a valence of 2. These inorganic oxides may be added to the catalyst at any suitable point.

In one or more embodiments, a catalytically active metal may be incorporated into the nanocrystalline zeolite catalyst by, for example, ion-exchange or impregnation of the zeolitic material, or by incorporating the active metal in the synthesis materials from which the zeolitic material is prepared. The catalytically active metal may be incorporated in the framework of the zeolitic material of the nanocrystalline zeolite catalyst, incorporated into channels of the zeolitic material of the nanocrystalline zeolite catalyst (i.e., occluded), or combinations thereof.

The catalytically active metal may be in a metallic form, combined with oxygen (e.g., metal oxide) or include derivatives of the compounds described below, for example. Suitable catalytically active metals depend upon the particular method in which the catalyst is intended to be used. Non-limiting examples of catalytically active metal that can be incorporated with the nanocrystalline zeolite catalyst can include lanthanum, cerium, yttrium, or a rare earth of the lanthanide series.

In one or more embodiments, the zeolitic material may include less than about 0.001 wt. % sodium, for example. In one or more embodiments, the zeolitic material may have a SiO2:Al2O3 ratio of greater than 7, for example. In one or more embodiments, the zeolitic material may include 0.1 to 0.8 Ce atoms per Al atom for example.

In one or more embodiments, the nanocrystalline zeolite catalyst may be formed by utilizing a carrier to transport the zeolitic material into the pores of the support material. Support materials are well known in the art and possess well-arranged pore systems with uniform pore sizes; however, support materials tend to possess either only micropores or only mesopores, in most cases only micropores. Micropores are defined as pores having a diameter of less than about 2 nm. Mesopores are defined as pores having a diameter ranging from about 2 nm to about 50 nm. Micropores generally limit external molecules access to the catalytic active sites inside of the micropores or slow down diffusion to the catalytic active sites.

In one or more embodiments, the carrier may have nano-sized particles (with the nano-sized particles of the carrier defined for use with nano-sized particles of the zeolitic material).

The formed zeolite may then be dried, for example. It is further contemplated that the carrier may be mixed with a solvent prior to contact with the nanocrystalline zeolite.

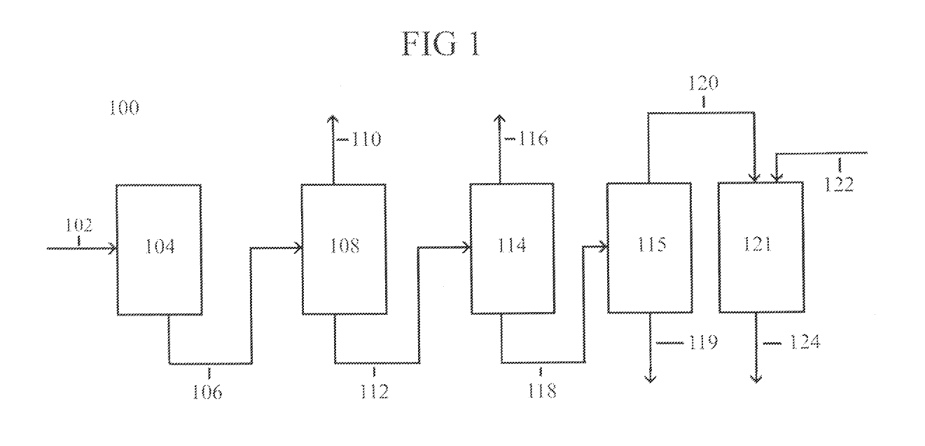

FIG. 1 illustrates a flow diagram of an embodiment of a process 100 for aromatic conversion utilizing a nanocrystalline zeolite catalyst that can decrease the amount of polyalkyl aromatic hydrocarbons in a product stream. While the majority of embodiments discussed herein relate to the aromatic conversion of benzene to ethylbenzene, it should be understood embodiments may include conversion of other compounds, such as aromatic conversion of benzene to cumene, for example.

As shown, the process 100 may include a variety of feed streams. Feed stream 102 may contain benzene, the alkylatable aromatic hydrocarbon and may contain ethylene, the acyclic alkene. In one or more embodiments, feed stream 102 is in the liquid phase. Suitable aromatic hydrocarbons in feed stream 102 include benzene, naphthalene, anthracene, naphthacene, perylene, coronene, and phenanthrene, with benzene being preferred in one or more embodiments.

In one or more embodiments, feed stream 102 may include alkyl substituted aromatic hydrocarbons. Suitable alkyl substituted aromatic compounds include toluene, xylene, isopropylbenzene, normal propylbenzene, alpha-methylnaphthalene, ethylbenzene, mesitylene, durene, cymenes, butylbenzene, pseudocumene, o-diethylbenzene, m-diethylbenzene, p-diethylbenzene, isoamylbenzene, isohexylbenzene, pentaethylbenzene, pentamethylbenzene; 1,2,3,4-tetraethylbenzene; 1,2,3,5-tetramethylbenzene; 1,2,4-triethylbenzene; 1,2,3-trimethylbenzene, m-butyltoluene; p-butyltoluene; 3,5-diethyltoluene; o-ethyltoluene; p-ethyltoluene; m-propyltoluene; 4-ethyl-m-xylene; dimethylnaphthalenes; ethylnaphthalene; 2,3-dimethylanthracene; 9-ethylanthracene; 2-methylanthracene; o-methylanthracene; 9,10-dimethylphenanthrene; and 3-methyl-phenanthrene. Higher molecular weight alkylaromatic hydrocarbons may also be used as starting materials and include aromatic hydrocarbons such as are produced by the alkylation of aromatic hydrocarbons with olefin oligomers. Such products are frequently referred to in the art as alkylate and include hexylbenzene, nonylbenzene, dodecylbenzene, pentadecylbenzene, hexyltoluene, nonyltoluene, dodecyltoluene, pentadecytoluene, etc. Very often alkylate is obtained as a high boiling fraction in which the alkyl group attached to the aromatic nucleus varies in size from about C₆ to about C₁₂.

In one or more embodiments, benzene is fed to an alkylation reactor 104 along with ethylene, which is an acyclic alkene. The reactor 104 contains an alkylation catalyst. The benzene and ethylene contact the alkylation catalyst in the reactor 104 and react to form a ethylbenzene, a mono alkyl aromatic hydrocarbon. Further process equipment may include, in addition to the alkylation reactor 104, separators 108, 114, 115 and a transalkylation reactor 121, for example.

FIG. 1 illustrates a schematic block diagram of an embodiment of an alkylation/transalkylation process 100. The process 100 generally includes supplying an input stream 102 (e.g., a first input stream) to an alkylation system 104 (e.g., a first alkylation system.) The alkylation system 104 is generally adapted to contact the input stream 102 with an alkylation catalyst to form an alkylation output stream 106 (e.g., a first output stream).

At least a portion of the alkylation output stream 106 passes to a first separation system 108. An overhead fraction is generally recovered from the first separation system 108 via line 110 while at least a portion of the bottoms fraction is passed via line 112 to a second separation system 114.

An overhead fraction is generally recovered from the second separation system 114 via line 116 while at least a portion of a bottoms fraction is passed via line 118 to a third separation system 115. A bottoms fraction is generally recovered from the third separation system 115 via line 119 while at least a portion of an overhead fraction is passed via line 120 to a transalkylation system 121. In addition to the overhead fraction 120, an additional input, such as additional aromatic compound, is generally supplied to the transalkylation system 121 via line 122 and contacts the transalkyation catalyst, forming a transalkylation output 124.

Although not shown herein, the process stream flow may be modified based on unit optimization. For example, at least a portion of any overhead fraction may be recycled as input to any other system within the process. Also, additional process equipment, such as heat exchangers, may be employed throughout the processes described herein and placement of the process equipment can be as is generally known to one skilled in the art. Further, while described in terms of primary components, the streams indicated may include any additional components as known to one skilled in the art.

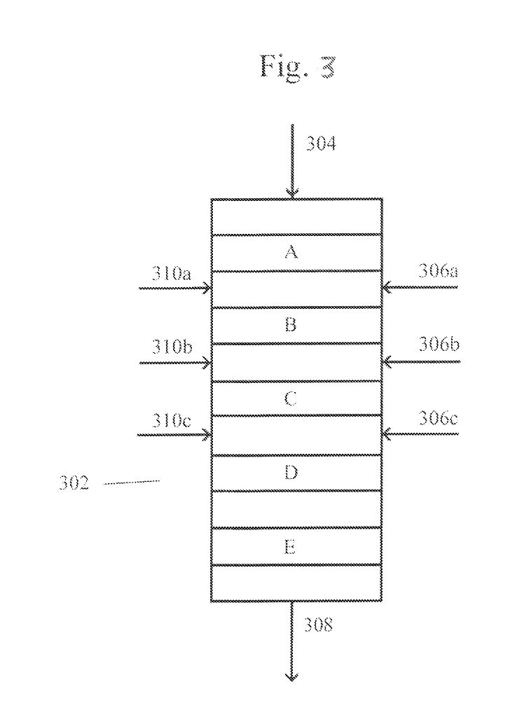

The input stream 102 generally includes an aromatic compound and an alkylating agent. The aromatic compound may include substituted or unsubstituted aromatic compounds. The aromatic compound may include hydrocarbons, such as benzene, for example. If present, the substituents on the aromatic compounds may be independently selected from alkyl, aryl, alkaryl, alkoxy, aryloxy, cycloalkyl, halide and/or other groups that do not interfere with the alkylation reaction, for example. The aromatic compound and an alkylating agent can be input at multiple locations, such as in an embodiment as shown in FIG. 3.

The alkylating agent may include olefins such as ethylene or propylene, for example. In one embodiment, the aromatic compound is benzene and the alkylating agent is ethylene, which react to form a product that includes ethylbenzene as a significant component, for example.

The alkylation system 104 can include a plurality of multi-stage reaction vessels. In one embodiment, the multi-stage reaction vessels can include a plurality of operably connected catalyst beds containing an alkylation catalyst. An example of a multi-stage reaction vessel is shown in FIG. 3. Such reaction vessels are generally liquid phase reactors operated at reactor temperatures and pressures sufficient to maintain the alkylation reaction in the liquid phase, i.e., the aromatic compound is in the liquid phase. Such temperatures and pressures are generally determined by individual process parameters. For example, the reaction vessel temperature may be from 65° C. to 300° C., or from 200° C. to 280° C., for example. The reaction vessel pressure may be any suitable pressure in which the alkylation reaction can take place in the liquid phase, such as from 300 psig to 1,200 psig, for example.

In one embodiment, the space velocity of the reaction vessel within the alkylation system 104 is from 10 to 200 hr⁻¹ liquid hourly space velocity (LHSV) per bed, based on the aromatic feed rate. In alternate embodiments, the LHSV can range from 10 to 100 hr⁻¹, or from 10 to 50 hr⁻¹, or from 10 to 25 hr⁻¹ per bed. For the alkylation process overall, including all of the alkylation beds of the preliminary alkylation reactor or reactors and the primary alkylation reactor or reactors, the space velocity can range from 1 to 20 hr⁻¹ LHSV.

The alkylation output 106 generally includes a second aromatic compound. In one embodiment, the second aromatic compound includes ethylbenzene, for example.

A first separation system 108 may include any process or combination of processes known to one skilled in the art for the separation of aromatic compounds. For example, the first separation system 108 may include one or more distillation columns (not shown) either in series or in parallel. The number of such columns may depend on the volume of the alkylation output 106 passing through.

The overhead fraction 110 from the first separation system 108 generally includes the first aromatic compound, such as benzene, for example.

The bottoms fraction 112 from the first separation system 108 generally includes the second aromatic compound, such as ethylbenzene, for example.

A second separation system 114 may include any process known to one skilled in the art, for example, one or more distillation columns (not shown), either in series or in parallel.

The overhead fraction 116 from the second separation system 114 generally includes the second aromatic compound, such as ethylbenzene, which may be recovered and used for any suitable purpose, such as the production of styrene, for example.

The bottoms fraction 118 from the second separation system 114 generally includes heavier aromatic compounds, such as polyethylbenzene, cumene and/or butylbenzene, for example.

A third separation system 115 generally includes any process known to one skilled in the art, for example, one or more distillation columns (not shown), either in series or in parallel.

In a specific embodiment, the overhead fraction 120 from the third separation system 115 may include diethylbenzene and triethylbenzene, for example. The bottoms fraction 119 (e.g., heavies) may be recovered from the third separation system 115 for further processing and recovery (not shown).

The transalkylation system 121 generally includes one or more reaction vessels having a transalkylation catalyst disposed therein. The transalkylation catalyst can include nanocrystalline zeolite catalyst. The reaction vessels may include any reaction vessel, combination of reaction vessels and/or number of reaction vessels (either in parallel or in series) known to one skilled in the art.

A transalkylation output 124 generally includes the second aromatic compound, for example, ethylbenzene. The transalkylation output 124 can be sent to one of the separation systems, such as the second separation system 114, for separation of the components of the transalkylation output 124.

In one embodiment, the transalkylation system 121 is operated under liquid phase conditions. For example, the transalkylation system 121 may be operated at a temperature of from about 65° C. to about 290° C. and a pressure of about 800 psig or less.

In a specific embodiment, the input stream 102 includes benzene and ethylene. The benzene may be supplied from a variety of sources, such as for example, a fresh benzene source and/or a variety of recycle sources. As used herein, the term “fresh benzene source” refers to a source including at least about 95 wt % benzene, at least about 98 wt % benzene or at least about 99 wt % benzene, for example. In one embodiment, the molar ratio of benzene to ethylene may be from about 1:1 to about 30:1, or from about 1:1 to about 20:1, for the total alkylation process including all of the alkylation beds, for example. The molar ratio of benzene to ethylene for individual alkylation beds can range from 10:1 to 100:1, for example.

In a specific embodiment, benzene is recovered through line 110 and recycled (not shown) as input to the alkylation system 104, while ethylbenzene and/or polyalkylated benzenes are recovered via line 112.

As previously discussed, the alkylation system 104 generally includes an alkylation catalyst that can include nanocrystalline zeolite catalyst. The input stream 102, e.g., benzene/ethylene, contacts the alkylation catalyst during the alkylation reaction to form the alkylation output 106, e.g., ethylbenzene.

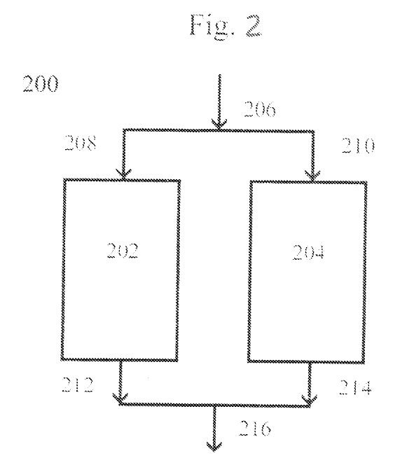

FIG. 2 illustrates a non-limiting embodiment of an alkylation system 200. The alkylation system 200 shown includes a plurality of alkylation reactors, such as two alkylation reactors 202 and 204, operating in parallel. One or both alkylation reactors 202 and 204, which may be the same type of reaction vessel, or, in certain embodiments, may be different types of reaction vessels, may be placed in service at the same time. For example, only one alkylation reactor may be on line while the other is undergoing maintenance, such as catalyst regeneration. In one embodiment, the alkylation system 200 is configured so that the input stream 206 is split to supply approximately the same input to each alkylation reactor 202 and 204. However, such flow rates will be determined by each individual system.

By way of example, during normal operation of the system 200, with both reactors 202 and 204 on-line, the input stream 206 may be supplied to both reactors (e.g., via lines 208 and 210) to provide a space velocity that is less than if the entire input stream 206 was being sent to a single reactor. The output stream 216 may be the combined flow from each reactor (e.g., via lines 212 and 214). When a reactor is taken off-line and the feed rate continues unabated, the space velocity for the remaining reactor may approximately double.

In a specific embodiment, one or more of the plurality of alkylation reactors may include a plurality of interconnected catalyst beds. The plurality of catalyst beds may include from 2 to 15 beds, or from 5 to 10 beds or, in specific embodiments, 5 or 8 beds, for example. Embodiments can include one or more catalyst beds having a mixed catalyst load that includes a nanocrystalline zeolite catalyst and one or more other catalyst. The mixed catalyst load can, for example, be a layering of the various catalysts, either with or without a barrier or separation between them, or alternately can include a physical mixing where the various catalysts are in contact with each other.

FIG. 3 illustrates a non-limiting embodiment of an alkylation reactor 302. The alkylation reactor 302 includes five series connected catalyst beds designated as beds A, B, C, D, and E. An input stream 304 (e.g., benzene/ethylene) is introduced to the reactor 302, passing through each of the catalyst beds to contact the alkylation catalyst and form the alkylation output 308. Additional alkylating agent may be supplied via lines 306a, 306b, and 306c to the interstage locations in the reactor 302. Additional aromatic compound may also be introduced to the interstage locations via lines 310a, 310b and 310c, for example. One or more of the catalyst beds can contain nanocrystalline zeolite catalyst.

The processes described herein (and particularly the catalysts described herein in combination with the described methods) are capable of reducing byproduct formation, such as reduced polyethylbenzene, in a reactor containing the inventive alkylation catalyst.

Each reactor of the process may include more than one reactor connected in parallel or in series, where each reactor contains at least one reaction zone and at least one alkylation catalyst in the reaction zone.

Reactors 104 and 121 may be capable of operation at elevated temperatures and pressures required for aromatic conversion, and may be capable of enabling contact of the reactants (e.g., benzene and ethylene) with the inventive alkylation catalyst. Specific embodiments of the particular reactors 104 and 121 may be determined based on the particular design conditions and throughput by one of ordinary skill in the art, and are not meant to be limiting on the scope of the disclosed method.

The operating conditions of the alkylation reactor 104 may be system specific and may vary depending on the composition of the feed stream 102 and the composition of the product stream 106. In one or more embodiments, the reactor(s) may operate at elevated temperatures and pressures, for example. In one or more embodiments, the elevated temperature may range from about 100° C. to about 500° C., or from about 160° C. to about 480° C., or from about 170° C. to about 460° C., for example. The elevated pressure may range from about 10 atm to about 70 atm, or from about 10 atm to about 50 atm, or from about 10 atm to about 35 atm, for example.

In one or more embodiments, the transalkylation reaction takes place under liquid phase conditions. Particular conditions for carrying out the liquid phase transalkylation of poly aromatic hydrocarbons with benzene may include a temperature of from about 150° C. to about 280° C., a pressure of about 101 psia to about 600 psia, and a mole ratio of benzene to polyalkyl aromatic hydrocarbons of from about 1:1 to about 30:1, or from about 1:1 to about 10:1, and or from about 1:1 to about 5:1, for example.

In one or more embodiments, the reaction zone(s) of reactors 104 and 121 may include one or more catalyst beds. The catalyst beds may include fixed bed, fluidized beds, entrained beds or combinations thereof, for example. When utilizing multiple beds, an inert material layer may separate each bed. The inert material may include any type of inert substance, such as quartz, for example. In one or more embodiments, the reactors 104 and 121 may include from one to ten catalyst beds or from two to five catalyst beds, for example.

It is contemplated that the inventive alkylation catalyst may be used in any number of catalyst beds present in the process 100.

The nanocrystalline zeolite catalyst described herein increases the effective surface area of the catalyst and provides smaller pore volumes which can reduce the formation of polyethylbenzenes by limiting the contact time on the active catalyst surface and reducing the contact time such that it does not reach the equilibrium of diethylbenzene, thus reducing its formation and providing a product stream with fewer undesirable components.

The nanocrystalline zeolite catalyst can have an increased ratio of surface area to volume due to the particle size of the zeolitic material compared to zeolitic materials that are not nanocrystalline.

Because limiting the contact time on the active catalyst surface with the nanocrystalline zeolite catalyst, amounts of polyalkyl aromatic hydrocarbons is reduced. Thus, the size of the transalkylation reactor 121 may be reduced, and in some operating conditions, a transalkylation reactor 121 may not be needed altogether. Either scenario reduces capital cost, operating cost, and maintenance cost associated with the disclosed method 100 over traditional alkylation processes.

In an embodiment a method for aromatic conversion includes contacting an alkene and an aromatic hydrocarbon with a nanocrystalline zeolite catalyst disposed within a reactor under alkylation conditions, wherein the nanocrystalline zeolite catalyst comprises at least one zeolitic material and producing a product stream having a monoalkyl aromatic hydrocarbon.

In an embodiment the selectivity for the monoalkyl aromatic hydrocarbon is at least 92 mass percent, optionally at least 95 mass percent, or optionally at least 97 mass percent.

In an embodiment the product stream has less than 5 mass percent of a polyalkyl aromatic hydrocarbon, optionally less than 4 mass percent, or optionally less than 3 mass percent.

The various aspects of the present invention can be joined in combination with other aspects of the invention and the listed embodiments herein are not meant to limit the invention. All combinations of various aspects of the invention are enabled, even if not given in a particular example herein.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the aspects and embodiments disclosed herein are usable and combinable with every other embodiment and/or aspect disclosed herein, and consequently, this disclosure is enabling for any and all combinations of the embodiments and/or aspects disclosed herein. Other and further embodiments, versions and examples of the invention may be devised without departing from the basic scope thereof and the scope thereof is determined by the claims that follow.

While the foregoing is directed to embodiments of the present invention, other and further embodiments of the invention may be devised without departing from the basic scope thereof and the scope thereof is determined by the claims that follow.

What is claimed is:

1. A method for aromatic conversion comprising:
contacting an alkene and an aromatic hydrocarbon with a nanocrystalline zeolite catalyst disposed within a reactor under alkylation conditions, wherein the nanocrystalline zeolite catalyst comprises at least one zeolitic material; and
producing a product stream having a monoalkyl aromatic hydrocarbon.

2. The method of claim 1, wherein the nanocrystalline zeolite catalyst has a particle size of 600 nm or less.

3. The method of claim 2, wherein the nanocrystalline zeolite catalyst has a particle size of less than about 300 nm.

4. The method of claim 1, wherein the nanocrystalline zeolite catalyst comprises a molecular sieve.

5. The method of claim 1, wherein the nanocrystalline zeolite catalyst is selected from the group consisting of zeolite Y, rare earth exchanged zeolite Y, zeolite X, rare earth exchanged zeolite X, MCM-22, MCM-36, MCM-49, zeolite beta, ZSM-4, ZSM-12, ZSM-20, ZSM-38, MOR zeolite framework, OFF zeolite framework, LTL zeolite framework, and any combination thereof.

6. The method of claim 1, wherein the nanocrystalline zeolite catalyst has a framework silica to alumina molar ratio of between about 2:1 to about 300:1.

7. The method of claim 1, wherein the nanocrystalline zeolite catalyst has a framework silica to alumina molar ratio of between about 5:1 to about 200:1.

8. The method of claim 1, further comprising:
incorporating a catalytically active metal into the zeolitic material of the nanocrystalline zeolite catalyst.

9. The method of claim 8, wherein the catalytically active metal is selected from the group consisting of lanthanum, cerium, yttrium, a rare earth of the lanthanide series, and any combination thereof.

10. The method of claim 8, further comprising:
contacting the catalytically active metal with a carrier prior to the step of incorporating.

11. The method of claim 8, further comprising:
contacting the zeolitic material with a carrier prior to the step of incorporating.

12. The method of claim 1, wherein the nanocrystalline zeolite catalyst further comprises a support material combined with the zeolitic material.

13. The method of claim 12, wherein the support material is selected from the group consisting of silica, alumina, aluminosilica, titania, zirconia, silicon carbide, and any combination thereof.

14. The method of claim 12, wherein the nanocrystalline zeolite catalyst comprises from about 5 wt. % to about 95 wt. % support material.

15. The method of claim 12, further comprising:
transporting the nanocrystalline zeolite catalyst into the pores of the support material with a carrier.

16. The method of claim 1, wherein the aromatic hydrocarbon comprises benzene, wherein the alkene comprises ethylene, wherein the monoalkyl aromatic hydrocarbon comprises ethylbenzene.

17. The method of claim 1, wherein the aromatic hydrocarbon comprises benzene, wherein the alkene comprises propene, wherein the monoalkyl aromatic hydrocarbon comprises cumene.

18. The method of claim 1, wherein the selectivity for the monoalkyl aromatic hydrocarbon is at least about 92 mass percent.

19. The method of claim 1, wherein the product stream further comprises less than about 5 mass percent of a polyalkyl aromatic hydrocarbon.

20. A method for aromatic conversion comprising:
contacting an alkene and an aromatic hydrocarbon with a molecular sieve having a nanocrystalline zeolite catalyst component disposed within a reactor under alkylation conditions; and
producing a product stream having a monoalkyl aromatic hydrocarbon;
wherein the nanocrystalline zeolite catalyst has a particle size of 600 nm or less;
wherein the molecular sieve has a framework silica to alumina molar ratio of between about 5:1 to about 30:1 and comprises a zeolitic material selected from the group consisting of zeolite Y, rare earth exchanged zeolite Y, zeolite X, rare earth exchanged zeolite X, MCM-22, MCM-36, MCM-49, zeolite beta, ZSM-4, ZSM-12, ZSM-20, ZSM-38, MOR zeolite framework, OFF zeolite framework, LTL zeolite framework, and any combination thereof;
wherein the nanocrystalline zeolite catalyst further comprises a support material selected from the group consisting of silica, alumina, aluminosilica, titania, zirconia, silicon carbide, and any combination thereof combined with the zeolitic material;
wherein the nanocrystalline zeolite catalyst comprises from about 5 wt. % to about 95 wt. % support material.
